(19) United States
(12) Patent Application Publication
(10) Pub. No.: US 2015/0025356 A1
(43) Pub. Date: Jan. 22, 2015

(54) ASSESSMENT METHOD

(71) Applicant: Catherine Gibson, (US)

(72) Inventor: David John Michael Gibson, Wellington (NZ)

(21) Appl. No.: 14/129,037

(22) PCT Filed: Jul. 2, 2012
(86) PCT No.: PCT/NZ2012/000115

§ 371 Date: Aug. 26, 2014

(30) Foreign Application Priority Data
Jul. 1, 2011 (NZ) 593857

(51) Int. Cl. A01K 29/00; A61B 5/00

(52) U.S. Cl. CPC A01K 29/005; A61B 5/4848
USPC 600/407; 600/595

(57) ABSTRACT

A method of assessing the well being of an animal is disclosed. The method comprises obtaining information as to movement characteristics of the animal before applying medical treatment to the animal; obtaining information as to movement characteristics of the animal after applying medical treatment to the animal; comparing the movement characteristics of the animal before applying the medical treatment with the movement characteristics of the animal after applying the medical treatment and identifying differences between these; and assessing the differences and, based on these, determining whether the well being of the animal is improving as a result of the treatment.

### FIELD OF THE INVENTION

A preferred form of the present invention relates to a method of assessing the well being of an animal. More particularly but not exclusively the present invention relates to a method of assessing differences in movements of an animal before and after applying medical treatment to determine whether the health of the animal is improving as a result of the treatment.

### BACKGROUND TO THE INVENTION

There exists in the prior art different methods and systems for performing a diagnosis and/or prognosis of medical conditions affecting animals as well as humans. A medical diagnosis is a method of identifying a condition that may be affecting a subject. The steps include a physical examination for symptoms, medical tests such as blood tests and imaging, and when possible a description of symptoms by the subject.

Following a diagnosis, a prognosis may be provided which attempts to predict the expected outcome or course of the condition. A prognosis may take into account the condition of the subject, the severity of the condition and the expected effect of treatment. The prognosis may be based on statistical data collected from many prior cases and/or the experience or judgement of a medical practitioner. At any time, a new prognosis may be provided based on re-evaluating the same factors if the condition of the subject has changed. These known methods use the condition of the subject as the main factor for providing a prognosis rather than the measured effect of a treatment.

### OBJECT OF THE INVENTION

It is an object of the invention to provide a method for assessing the well being of an animal; and/or to at least provide the public with a useful choice.

### SUMMARY OF THE INVENTION

According to one aspect of the invention there is provided a method of assessing the well being of an animal comprising:
a) obtaining information as to movement characteristics of the animal before applying medical treatment to the animal;
b) obtaining information as to movement characteristics of the animal after applying medical treatment to the animal;
c) comparing the movement characteristics of the animal before applying the medical treatment with the movement characteristics of the animal after applying the medical treatment and identifying differences between these; and
d) assessing the differences and, based on these, determining whether the well being of the animal is improving as a result of the treatment.

Preferably the comparison of differences in the movement characteristics of the animal is used to determine whether the medical treatment should be altered.

Preferably the differences are used to determine a trend and, based on the trend, to predict a future level of well being of the animal based on projected continuation of the medical treatment.

Preferably deviations from the trend are used to determine if the medical treatment applied to the animal should be altered.

Preferably sensing means is used to capture information indicative of the movement characteristics.

Preferably the sensing means comprises a camera or motion sensor.

Preferably the comparison of differences is used to diagnose a health condition of the animal.

Preferably the animal suffers from arthritis and the medical treatment is for addressing arthritis.

Preferably the medical treatment comprises administration of a drug.

Preferably the medical treatment comprises administration of physical therapy.

Preferably the medical treatment comprises a diet change.

According to a further aspect of the invention there is provided a method of assessing the well being of an animal as set out above by way of a computer system comprising:
a) obtaining information using sensing means as to movement characteristics of the animal before applying medical treatment to the animal;
b) obtaining information using the sensing means as to movement characteristics of the animal after applying medical treatment to the animal;
c) conveying the information obtained before and after medical treatment as data to the computer system by way of data transfer means;
d) the computer system processing the data to compare differences in the movement characteristics of the animal between before and after applying medical treatment; and
e) the computer system assessing the differences and, based on these, determining whether the well being of the animal is improving as a result of the treatment.

### BRIEF DESCRIPTION OF THE FIGURES

Preferred forms of the invention will now be described with reference to the accompanying figures in which:

FIG. 1 shows a graph of measurements relating to daily physical activity of a subject before and after treatment.

### DETAILED DESCRIPTION OF THE PREFERRED EMBODIMENTS

In reference to the figure, the preferred embodiments of the invention will be illustrated by way of non-limiting example.

Although it is anticipated that the general invention as described has other uses, the preferred use of the invention is a method of assessing the treatment of a health condition.

The invention may be used for assessing the state of a health condition of an animal, and any subsequent change in that health condition following the commencement of treatment. The initial state of the health condition, along with initial improvements following treatment or a continuation of a treatment is used to establish a treatment model that predicts the course of the treatment. The treatment model, is amended and/or refined as the course continues, in response to further assessments of the health condition. The treatment course may also be altered in response to established deviations from the treatment model. The treatment model may be based on one or more measurable attributes relating to the condition.

The invention may also be used as part of a diagnosis, wherein if a favourable response follows treatment, then the type of successful treatment may be indicative of the nature of the condition.

### Example 1

In this embodiment, the invention relates to a method for assessing and developing a treatment model for the treatment of a condition affecting the movement or physical activity of a subject. A particular example is arthritis, which exists in many forms and affects a wide range of animals. Arthritis also affects humans. For the purposes of this specification the term “animal” does not include “human”.

The methods of the invention may follow a diagnosis of arthritis, or may be used as part of a diagnosis by determining if any improvements result from a treatment expected to ameliorate any arthritis symptoms. This embodiment may be useful for any condition that may be subject to change, or may be responsive to treatment and has an effect of the movements, mobility, or general physical activity of the subject.

To determine a baseline assessment of the condition, a monitoring means is used that can measure the physical activity of the subject. Depending on factors such as the type of condition and the circumstances of the subject, the monitoring means may provide a measurement of overall physical activity, general mobility, particular body or limb movements, or any combination thereof. Any suitable monitoring means may be used, for instance cameras, or motion sensors worn by the subject.

A physical activity measurement may provide several advantages over other methods of assessing a condition. For instance a traditional assessment for arthritis may include a questionnaire to diagnose the disorder and evaluate the severity of symptoms. This may introduce undesired subjectivity into the assessment due either in the responses of the subject, or the interpretation of a physician. Additionally, such a questionnaire is only suitable for a human subject who is able to suitably understand and respond to the questions. Another known method for assessing arthritis is testing for biomarkers in blood samples. These biomarkers may be useful in determining general disease states and differentiating between forms of arthritis, however they are not all conclusive and may not be indicative or directly proportional to the actual level of discomfort, pain or severity of other symptoms experienced by the subject. In some cases the change in a bio-marker may also not be commensurate with a change in the symptoms.

A measurement of physical activity can be used to assess the general well being and effect of a particular condition on a subject. As levels of pain, discomfort or other effects of arthritis such as tiredness, muscle weakness, and joint stiffness decrease, it is expected that the level of physical activity will increase. Such an objective measurement of the reduction of symptoms may be used to assess the progress of a treatment or a condition.

Different measurements of physical activity may be performed on the subject depending on the type of condition or the characteristics of the subject. For instance, a general measure of all of the subject's movements may provide a suitable means of assessment. This may be a measure of the general movements of the subject's body, or it may be a measure of the subject's apparent mobility by determining changes in location.

In other cases it may be more appropriate to make more specific measurements, such as the activity of one or more limbs that are affected by the condition, or a comparison of one affected limb to one non-affected limb. Any specified movement or type of movement may be suitable.

The physical activity measurements used for an assessment may be additionally refined or filtered to provide more meaningful or useful results. For instance, a measurement of physical activity as part of an assessment of a condition that causes pain or discomfort, may be more correlated to the level of pain or discomfort at times when the subject is not compelled to perform any physical activity. If an exercise regime comprises part of the course of treatment provided to the subject, it may be useful to exclude or provide a different interpretation to those periods of time.

An assessment using the measured physical activity of the subject may be made before any treatment commences, or at any time after or during treatment. Once a baseline assessment has been made, a subsequent assessment is made following a period of time in which a suitable treatment may have been provided.

A change in the condition of the subject may be established from the subsequent assessment. Using the initial assessment and at least one further assessment, a treatment model can be established that predicts the condition of the subject over the course of treatment.

Any deviations from the predicted state of the subject's condition can be determined from subsequent assessments. These deviations may require altering the treatment provided to the subject. For instance, a reduction in the rate of improvement of a condition may necessitate altering the treatment course, such as by increasing the administration of a drug. However, a reduction in the rate of improvement may indicate a plateau or limit to the effect of the treatment. Other forms of treatment include altering the diet of the animal or administering physical therapy to the animal.

The level of physical activity of the subject will have a maximum level, dependent on factors such as the age of the subject, behavioural characteristics, and any limitations the course of treatment may have on the condition. When a maximum level has been reached it may be that the condition has been completely resolved by the treatment, or that the condition can no longer be ameliorated by the treatment. A different course of action may then be taken, such as discontinuing the treatment, continuing the treatment at a maintenance level, or beginning a new course of treatment. To establish a required maintenance level, further assessments may be made in accordance with the invention to monitor the effect of changes in the treatment to determine the minimum required treatment to maintain the desired level of physical activity.

It may also be useful or necessary to use additional measurements, such as bio-markers from blood tests, to assess other factors relating to the condition or treatment. For instance side effects of a treatment may not be apparent from movement or other physical activity data, but may be measured with blood tests. In such cases a compromise may be required to find a balance between the maximum dosage of a treatment drug and the tolerable level of side effects.

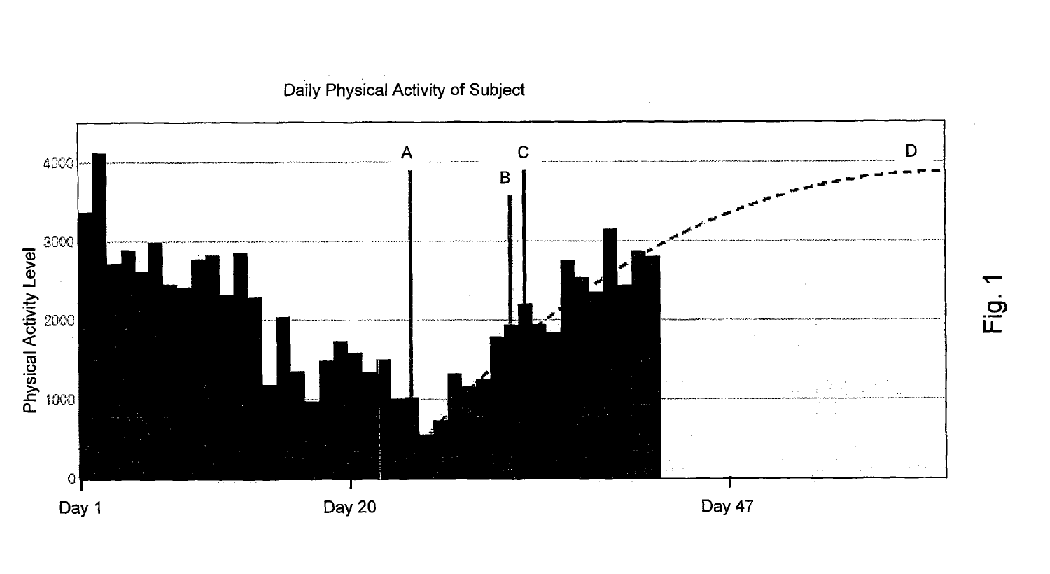

In a specific example of use, the subject is a dog that has been diagnosed with arthritis. A graph shown in FIG. 1, displays a measure of daily total physical activity of the subject for a period of time after diagnosis. Each bar represents a summation of the measured physical activity for that day. The activity measurements made from day 1 to day 23 show an average decrease of daily physical activity due to the effects of arthritis. Treatment was commenced on day 24, comprising an NSAID injection ‘A’ in conjunction with a diet specified for joint mobility. Further NSAID injections ‘B’ and ‘C’ were administered on days 31 and 32 respectively. The increase in average physical activity provided sufficient data to form a prediction on the expected improvement in physical activity over course of treatment shown as a dashed line ‘0’. Deviations from this prediction can be determined by the physical activity measurements, allowing changes in the treatment to be made as necessary. The model predicts that the expected treatment time is 59 days.

The methods of assessing animals described above may be done by way of computer system. The computer system comprises one or more computers or other similar device(s). For example, information about the movements of the animal is obtained before and after applying medical treatment. This information is obtained using the sensors. The information is transferred as data to the computer system using data transfer means, for example a wireless transmitter. The computer system processes the data to compare differences in the movements of the animal between before and after applying medical treatment. The computer system then assesses the differences and determines from them whether the health of the animal is improving as a result of the treatment.

While some preferred forms of the invention have been described by way of example it should be appreciated that modifications and improvements can occur without departing from the scope of the appended claims.

1. A method of assessing the well being of an animal comprising:
a) obtaining information as to movement characteristics of the animal before applying medical treatment to the animal;
b) obtaining information as to movement characteristics of the animal after applying medical treatment to the animal;
c) comparing the movement characteristics of the animal before applying the medical treatment with the movement characteristics of the animal after applying the medical treatment and identifying differences between these; and
d) assessing the differences and, based on these, determining whether the well being of the animal is improving as a result of the treatment.

2. The method according to claim 1, wherein the comparison of differences in the movement characteristics of the animal is used to determine whether the medical treatment should be altered.

3. The method according to claim 1, wherein the differences are used to determine a trend and, based on the trend, to predict a future level of well being of the animal based on projected continuation of the medical treatment.

4. The method according to claim 3, wherein deviations from the trend are used to determine if the medical treatment applied to the animal should be altered.

5. The method according to claim 4, wherein sensing means is used to capture information indicative of the movement characteristics.

6. The method according to claim 5, wherein the sensing means comprises a camera or motion sensor.

7. The method according to claim 1, wherein the comparison of differences is used to diagnose a health condition of the animal.

8. The method according to claim 1, wherein the animal suffers from arthritis and the medical treatment is for addressing arthritis.

9. The method according to claim 1, wherein the medical treatment comprises administration of a drug.

10. The method according to claim 1, wherein the medical treatment comprises administration of physical therapy.

11. The method according to claim 1, wherein the medical treatment comprises a diet change.

12. A method of assessing the well being of an animal by way of a computer system comprising:
a) obtaining information using sensing means as to movement characteristics of the animal before applying medical treatment to the animal;
b) obtaining information using the sensing means as to movement characteristics of the animal after applying medical treatment to the animal;
c) conveying the information obtained before and after medical treatment as data to the computer system by way of data transfer means;
d) the computer system processing the data to compare differences in the movement characteristics of the animal between before and after applying medical treatment; and
e) the computer system assessing the differences and, based on these, determining whether the well being of the animal is improving as a result of the treatment.

13. (canceled)

14. (canceled)

15. The method according to claim 12, wherein the comparison of differences in the movement characteristics of the animal is used to determine whether the medical treatment should be altered.

16. The method according to claim 12, wherein the differences are used to determine a trend and, based on the trend, to predict a future level of well being of the animal based on projected continuation of the medical treatment

17. The method according to claim 12, wherein deviations from the trend are used to determine if the medical treatment applied to the animal should be altered.

18. The method according to claim 12, wherein sensing means is used to capture information indicative of the movement characteristics.

19. The method according to claim 12, wherein the sensing means comprises a camera or motion sensor.

20. The method according to claim 12, Wherein the comparison of differences is used to diagnose a health condition of the animal.

21. The method according to claim 12, wherein the animal suffers from arthritis and the medical treatment is for addressing arthritis.

22. The method according to claim 12, wherein the medical treatment comprises administration of a drug, administration of physical therapy, or a diet change.
